(19) United States
(12) Patent Application Publication
(10) Pub. No.: US 2025/0049294 A1
(43) Pub. Date: Feb. 13, 2025

(54) METHOD, SYSTEM AND TRAINED MODEL FOR IMAGE OPTIMIZATION FOR ENDOSCOPES

(71) Applicant: KARL STORZ SE & Co. KG, Tuttlingen (DE)

(72) Inventors: Sebastian WENZLER, Tuttlingen (DE); Sebastian WAGNER, Tuttlingen (DE); Simon HAAG, Tuttlingen (DE); Patricia GALUSCHKA, Tuttlingen (DE)

(73) Assignee: KARL STORZ SE & Co. KG, Tuttlingen (DE)

(21) Appl. No.: 18/723,006

(22) PCT Filed: Dec. 15, 2022
(86) PCT No.: PCT/EP2022/086135

§ 371 Date: Jun. 21, 2024

(30) Foreign Application Priority Data
Dec. 23, 2021 (DE) 10 2021 134 564.2

(51) Int. Cl. A61B 1/00; A61B 1/12

(52) U.S. Cl. CPC A61B 1/000096; A61B 1/00006; A61B 1/00043; A61B 1/12

(57) ABSTRACT

A method and a control system for image analysis and optimization of at least one image acquired at the distal end of an endoscope includes the following steps: acquiring image data by means of an image acquisition device of an endoscope; receiving the image data by a control unit, pre-analyzing at least a subset of the image data of one or more consecutive images by a control unit to determine at least one image structure; determining a quality value by means of a training data-based, preferably self-learning, module by comparing the at least one determined image structure with image structures of a reference database stored in a memory unit, and on the basis of the determined quality value, manually or automatically outputting control instructions from the control unit to a unit for activating image optimization.

### CROSS-REFERENCE TO RELATED APPLICATIONS

This application is the U.S. national stage of PCT/EP2022/086135 filed on Dec. 15, 2022, which claims priority of German Patent Application No. 10 2021 134 564.2 filed on Dec. 23, 2021, the contents of which are incorporated herein.

### TECHNICAL FIELD

The disclosure relates to a method for image analysis and image optimization, a control system for image optimization, a trained model and a computer program product for carrying out said method.

### BACKGROUND

Endoscopes are well-known medical devices for examining cavities in a body or technical cavities. A commonly used type of endoscope has an optical system at the distal end of the endoscope, i.e. the end of the endoscope facing the body, and is designed to acquire images and transmit them to the endoscope operator. Optionally, additional functions can be made available via a working channel.

Endoscopes are known to be used in minimally invasive surgical procedures. An example of this is laparoscopy. Here, the view of the field to be examined is a crucial prerequisite for the endoscope operator to be able to carry out a diagnosis, manipulation or operation safely and quickly. It is important to be able to reliably distinguish between different tissue structures in the body cavity in order to make a correct diagnosis or avoid complications. Optimal visibility is necessary for this purpose.

When intervening in a human or animal body, the field of view of an endoscope is very small and even the smallest contamination, such as blood splashes, tissue particles or deposits of steam, smoke or grease, can severely impair the operator's vision. For example, if high-frequency surgical assistance (HF surgery) is applied, thermally induced changes in tissue cells are carried out using electrical energy with the aim of stopping bleeding or sealing tissue. In this type of medical procedures, HF activation can generate tissue particles that severely impair the vision for the endoscope operator. If the operator's vision is impaired in this way by unintentional tissue contact or other contamination, the endoscope must be cleaned extracorporeally.

This extracorporeal cleaning of the endoscope at its distal end is sometimes undesirable, especially when complications arise and the endoscope operator has to interrupt their medical or diagnostic procedure. In addition, extracorporeal cleaning also requires a certain amount of time, which must be taken into account in the overall duration of an operation. For example, if complications arise due to the perforation of an artery, the bleeding must be stopped as quickly as possible and a very good vision must be ensured for the endoscope user. If bleeding cannot be stopped quickly, other surgical methods that involve open surgery may be necessary. This should be avoided at all costs in order to keep the intervention as minimal as possible for the patient.

A clear and precise visualization of the object field to be observed is of great importance in endoscopic procedures. The operator who follows the visualization on a monitor can be negatively influenced by sub optimal monitor settings or environmental parameters such as room illumination. If the operator wears glasses, this cannot usually be taken into account. However, if the view is obstructed due to the problems mentioned above, the endoscope operator may need to interrupt the procedure. Unnecessary interruptions and distractions during the endoscopic procedure can result in misjudgments or endanger the health of a patient on whom the endoscope has been inserted. Any disturbance of vision also prolongs the entire endoscopic procedure, thereby increasing costs and reducing efficiency.

### SUMMARY

It is an object of the disclosure to overcome the above-mentioned problems and to meet the needs of an operator of endoscopic procedures, wherein a suitable method for image analysis and image optimization, preferably with the aid of an endoscope cleaning module, is to be used. The operator should be disturbed as little as possible, be assisted optimally and an automatic image optimization method should be provided. Since image acquisition can no longer be carried out with the required quality due to contamination, even when using a powerful image acquisition device, there is initially a need for automatic detection of dirt or other interference factors. There is also a need for methods for correcting the detected interferences and/or improve the environmental parameters. There is also a need to provide a system and a computer program product that uses automation processes to increase safety during an endoscopic procedure for the user and patient.

On the basis of the disclosure, the above-mentioned objects are to be achieved better than in conventional methods, in particular for human or veterinary medical applications. The image analysis methods and systems according to the disclosure can be used to examine both cavities in a body and technical cavities such as pipelines or the like. The aim is to optimize visibility conditions and quality for the endoscope operator.

These objects are achieved with a method for image analysis and image optimization, a control system and a computer program product and a trained model according to the features of the independent claims. Preferred embodiments of the disclosure emerge from the dependent claims following the independent claims. According to a first aspect of the disclosure, there is provided a method for image analysis and image optimization of at least one image acquired at the distal end of an endoscope, comprising the following steps:
acquiring image data by means of an image acquisition device of an endoscope: receiving the image data by a control unit,
pre-analyzing at least a subset of the image data of one or more subsequent images by a control unit to determine at least one image structure;
determining a quality value by means of a training data-based, preferably self-learning, module, by comparing the at least one determined image structure with image structures of a reference database stored in a memory unit; and
based on the determined quality value, manually or automatically outputting control instructions from a control unit to a unit for activating image optimization.

At least the image analysis with the determination of the quality value and optionally also the pre-analysis can run automatically in the training data-based or training data-based self-learning module. When activated after manual approval, image optimization using the method mentioned above can ensure visibility conditions and visual quality during an operation, at least partially automatically. If the process is set up completely automatically without manual intervention, an operator no longer has to decide whether the conditions for activating a unit, such as an endoscope for image optimization, are met. By determining the quality value, automated image optimization can be provided quickly and efficiently and interruptions can be avoided.

The computer-implemented method comprises, as a first step, the acquisition of image data using an image acquisition device of an endoscope. The image acquisition device can include not only medical endoscopes but also endoscopes that are used in other situations to examine inaccessible cavities, such as in manual operations on pipes and shafts. Furthermore, instead of a mono camera, a stereo endoscope having two optical systems can be provided as an image acquisition device. The image acquisition device can receive the data from a control unit wirelessly, for example via radio or via a cable. The control unit can be provided either locally in the endoscope or camera head or decentrally in a network. The information or image data can be received or transmitted via Internet or another network such as an operating network or hospital network as well as a “remote” system such as cloud-based system. The various method steps of the computer-implemented method according to the disclosure can be carried out at different locations such as the endoscope itself, in a computer and/or on decentralized servers.

The control unit may contain elements of a computer such as one or more microprocessors or graphics processors. For example, a graphics processing unit (GPU) can display a 2D or, when a stereo endoscope is provided, a 3D image in real time to a user on a screen or via glasses. In addition, the control unit advantageously has a memory unit or is communicatively connected to a memory unit. The memory unit, which can be provided locally in the control unit or in a decentralized manner, e.g. via servers or networks, has at least one database. A database comprises at least one reference database with image data comprising image structures.

By pre-analyzing at least a subset of the image data, such as an image section, an image structure can be detected. In this preliminary analysis, object detection or actual data acquisition can advantageously be carried out. Preferably, the image center can be used as a subset of the image data of an image. If data is already present in memory or is made available to a database from an external data source, a comparison can be made with image data from the database.

The further method step of determining the quality value is advantageously carried out at least partially automatically by means of a training-based self-learning module. Using training data, a quality value can be determined by comparing the image structure determined in the preliminary analysis with a history of image structures from the reference database. This allows the data to be classified depending on the quality value.

The self-learning module can be based on machine learning and improve itself through training data and experience. In machine learning, data reduction can be done manually by a person performing what is called feature extraction so that only a subset of the available features are used for machine learning (pre-classification or supervised learning).

In a preferred embodiment, the self-learning module can use a so-called deep learning model, which can learn completely independently without human intervention and can preferably use “deep data” as input and works with a neural network. With deep learning, human intervention is no longer necessary and the neural network takes over the feature extraction or pre-classification in order to subsequently perform a quality-value-dependent classification.

Based on the determined quality value, control instructions can be manually or automatically output from a control unit to a unit for activating image optimization.

According to a preferred embodiment, a further database, namely a so-called parameter database for control instructions for further units such as a camera, light source, insufflator or a cleaning module, can be provided and comprises parameters adapted depending on the unit to be controlled. Possible parameters can be, for example, an emission spectrum or intensity for a light source, insufflation pressure, humidity content or gas exchange parameters for an insufflator or cleaning parameters for a cleaning module. The parameters can also be adjusted using the self-learning module and can optionally be based on deep learning in conjunction with a stochastic approximation algorithm (Stochastic Approximation Algorithms). In this way, an optimal cleaning result can be achieved and the surgeon's work is no longer impaired by contamination or other automatically detectable factors.

According to a preferred embodiment, the unit that can be activated for image optimization is a unit of the endoscope, wherein said unit is preferably a cleaning module that is designed to clean at least one distal window by means of at least one fluid.

The activatable cleaning module allows a defined flushing quantity or volume of gas or liquid with a predeterminable flushing pressure to be released manually or automatically for at least one distal window. For example, if critical contamination has been detected by the quality value within a predetermined number of seconds (e.g. 2 or 3 s), the decision to clean can be made automatically and the cleaning module can be informed to open a fluid valve for a predetermined volume of gas or liquid (e.g. carbon dioxide or sodium chloride). After activation, the control instruction to interrupt the process can also be given automatically, i.e. in order to close the fluid valve. A closing control signal prevents the operator from activating the cleaning activation for longer than necessary.

According to a preferred embodiment, the quality value depends on the detected brightness of the image and/or contamination of the at least one distal window, wherein in the step of determining the quality value by means of the self-learning module a classification into contamination probabilities and/or degree of contamination takes place.

Using this classification, any image with critical contamination can be classified. The classification history is stored in the memory unit and is available for further learning. The training methods of the self-learning module depend on the selected model to be trained or trained and the input data. The input or input layer of the model of the self-learning module is a subset or the entirety of the image data of an endoscopic image, which is usually present as a two- or three-dimensional matrix of pixels. Three-dimensional images can be provided by stereoscopes, e.g. 3D endoscopes with two CCD cameras at the distal end. A stereo measurement system can also provide information as input data for the model as to whether and which of the two distal windows is contaminated. The output or output layer of the trained model is preferably a probability of contamination.

According to a preferred embodiment, the self-learning module comprises a model with a neural network, wherein the input data are the image data acquired by the image acquisition device, which can be extracted as individual images, pixels and/or image sections; and wherein the output data comprises the probability of contamination or blindness of the image acquisition device.

Preclassification and classification take place between input and output. The self-learning module has at least one learning unit, which is designed as a model to be trained at the beginning of the process. The model can be based on the technology of artificial intelligence, in particular on machine learning or preferably on an artificial neural network. A preferred embodiment of a neural network is a convolutional neural network (CNN), which can optimally perform image classification. Basically, the system architecture of a classical CNN consists of one or more convolutional layers, followed by a pooling layer that discards superfluous information. In principle, this unit can be repeated as often as desired. A large number of repetitions are referred to as “Deep Convolutional Neural Networks”, which fall within the scope of deep learning. Other forms of machine learning or deep learning models can also be used as the system architecture of the self-learning and training data-based module, as long as they are suitable for performing image classification based on endoscope images with fast calculation speed and reliably, i.e. with low error rates of, for example, less than 3% and a true-positive rate of at least 97%.

In the example of a binary classifier, where the classification algorithms divide the input data into only two possible classes, in some cases the training-based models used may assign an analyzed image to an incorrect class, with a true positive rate or sensitivity of at least 97%, and preferably 98%. The true positive rate indicates the probability that contamination was actually detected.

According to a preferred embodiment, the training data comprises image structures stored in a memory unit and/or a collection of markers used to train the self-learning module, wherein a trained model of the self-learning module is trained to classify the image data into the following contamination-dependent database classes or categories:
1. uncontaminated images,
2. contaminated images that can be cleaned by a cleaning module; and
3. contaminated or marked images that can be optimized by modules other than the cleaning module.

In addition to detected image structures, markers can also be used for classification. Preferably, a distinction is made between at least two categories or database classes that depend on the level of contamination (example of a binary classifier: class 1 (not contaminated) and class 2 (contaminated)). The images classified as contaminated can be cleaned by a cleaning module or by other modules such as an insufflator to remove smoke. Furthermore, contamination can be divided into further categories or classes depending on the degree or type of contamination as follows:
a) slightly contaminated
b) more contaminated
c) very contaminated
d) contaminated with blood
e) contaminated with fat

Using labels and/or markers, it is possible to control which task the model of the self-learning module is trained with. Using the training data provided (e.g. images of objects to be examined such as tissue structures with or without contamination or smoke or condensate) and associated labels (the category of contamination or image deterioration), a model such as an artificial neural network can learn accordingly.

According to a preferred embodiment, the self-learning module is based on machine learning, wherein a pre-classification is carried out by an expert or several persons and the subsequent quality value-dependent classification is carried out by a neural network. The classification of digital images and the assessment of the quality of a partial image or the entire image represents a technical application of machine learning. The classification algorithms used also serve to support the technical purpose of image optimization. In addition, algorithms derived from the training data for the model of the self-learning module can be checked and corrected by at least one expert.

In this way, an additional assessment during pre-classification by at least one expert can optionally be used for monitoring. For this purpose, an individual or a team of experts can label raw data or training images such as surgical videos and surgical images via a suitable interface such as a user interface. For example, ImageNet can be used to label a database of endoscope images. The labeling of the endoscope images can, for example, be done via a web service, wherein a corresponding web labeling tool is provided for all operators involved. The endoscope image experts thus provide labels for training the model of the self-learning module, which indicate which classification or category the respective training image falls into. Finally, experts can override the model, especially in an open network, for example during the training or learning phase, in order to make corrections at an early stage.

It is understood that the term “open neural network” in the context of this disclosure, defines an artificial intelligence network, wherein a third party such as e.g. an expert, if necessary corrects the learning or training process, so that supervised machine learning (“supervised learning”) takes place. For example, if the training data consists of pairs of input data and output data, an external expert can tell the neural network the correct output for each input, thus minimizing possible errors and deviations during training. In contrast, the term “closed neural network” is understood in the context of this disclosure to mean a neural network that learns without being supervised (“unsupervised learning”). Corrections are made automatically, namely input data is divided into classes without any external influence. According to a preferred embodiment, the self-learning module has as a model to be trained or already trained a model with a neural network based on machine learning or deep learning, wherein the neural network is selected from the group comprising: an open neural network, a closed neural network, a single-layer neural network, a multi-layer feedforward network with hidden layers, a feedback neural network and combinations thereof.

The neural networks mentioned above are characterized by their learning ability, whereby they can learn a task such as classification based on training data. Neural networks can find image structures in image data and thus recognize patterns. Using artificial neural networks, preferably by classifying image data, essential features can be extracted and functional relationships between the image structures can be approximated so that a quality value can be determined.

Open or closed artificial intelligence networks can be used, using both supervised and unsupervised machine learning methods. Alternatively, a mix of these methods, a so-called “semi-supervised” learning process, can be used to keep the costs incurred by an expert low while maintaining smaller data sets compared to the unsupervised training of the self-learning module.

One possibility for monitoring by experts or users is to use labels to assign a quality value to surgical images or other suitable data sets, for example depending on the disturbance in image quality. Such experts, users or externally commissioned labeling teams have experience in assessing the quality of image data and can peruse the necessary expert knowledge. The generated labels can be used for training data for the self-learning module and thus for neural networks. Depending on the rate of labeled data, either a supervised or a semi-supervised learning process is used.

According to a preferred embodiment, the at least one image structure represents at least one object to be examined that is depicted in the acquired image: wherein a contamination, deterioration or smoke detection is based on a change in the image structure.

In the preliminary analysis, object detection can be achieved by comparing the image structures. In this way, objects, organs, tissue structures to be examined or other structures can be detected. In a further analysis step, a classification based on machine learning is carried out, whereby the trained model can detect when the detected image structure has changed, for example due to smoke, condensate or contamination or other disruptive factors. Based on the change, a smoke event can then be detected, for example, or a degree of contamination can be classified using the calculated quality value.

According to a preferred embodiment, in the method step of pre-analysis, an acquired image can be subdivided into image sections and/or an acquired image can be subdivided into individual regions based on pixels on the basis of analysis values, wherein the quality value can be determined for each of the image sections and/or image regions.

According to a preferred embodiment, the cleaning module is designed to activate a regionally targeted cleaning depending on a definable weighting of regions and/or depending on a permissible percentage of contamination, wherein preferably the region of the distal window that can be assigned to the image center can be cleaned in a targeted manner.

Based on the analysis of preferred regions or image sections, cleaning can be carried out specifically for these regions/sections, thus optimizing the efficiency of cleaning. The preferred region for cleaning is the image center, since this is usually where the object to be examined is located and therefore the user has a particular interest in providing an optimal image. If a stereo endoscope is provided, one or two distal windows and thus the associated image acquisition devices can be cleaned, depending on the level of contamination.

According to a preferred embodiment, after activation of the cleaning module, a pulse of a fluid jet with a duration of a few milliseconds to a maximum of 1000 ms and under high pressure (p) of a maximum of 3 bar is directed specifically for cleaning at least part of the distal window of the image acquisition device and/or at one or more distal illumination windows of light sources.

In this way, cleaning can be carried out specifically for certain parts or the entirety of the at least one optical window of an image acquisition device. Advantageously, the light source or illumination device is also cleaned in order to extend the service life and optimize the light output of the light source.

An advantage of the combination of high pressure and short fluid pulses is that the user of the endoscope is hardly disturbed, since the field of vision is only briefly obscured by the cleaning fluid, like a windshield wiper. In addition, the control instruction or a corresponding signal for cleaning can advantageously be communicated to another unit. For example, an insufflator can be informed that cleaning has been activated so that the insufflator can adapt to the associated pressure change.

According to a preferred embodiment, the unit that can be activated for image optimization is selected from the group comprising:
one or more light sources of the endoscope for changing the illuminance; at least one filter for optimizing a contrast: autofocus system for adjusting the sharpness of the image: color spectrum change unit: end lens heating: monitor: user-dependent and/or endoscope- or light guide-dependent customizable software; room illumination of the operating room and combinations thereof.

In this way, the image can also be advantageously optimized by other surgical units than one or more cleaning modules. For example, by changing the color spectrum, wavelength ranges can be controlled for heating in order to reduce or remove condensation detected by the self-learning module using image classification. If an autofocus system is activated as a unit, the sharpness of the image can be adjusted with optimized parameters as output from the trained model.

Even factors that do not directly affect the object being examined by the endoscope, such as the illumination in the operating room, can be used to optimize the image. For example, the contrast of the monitor can be improved by dimming the interference from room illumination accordingly.

According to a preferred embodiment, the determination of a quality value comprises a blindness value due to smoke or condensation, wherein the control unit compares acquired characteristics for smoke or condensation with characteristics of a collection of characteristics stored in the reference database.

The self-learning module is thus designed to determine visual impairment caused by smoke or condensation based on a so-called blindness value. In this way, interference caused by the visual impairment can be automatically detected and eliminated or at least reduced by activating a unit such as a heating unit or a cleaning module. In this way, the quality of the image information contained in the acquired image can be improved.

According to a preferred embodiment, the method further comprises a control instruction for activating a smoke evacuator and/or an insufflator or a flushing pump in order to optimize the image by means of smoke evacuation; and/or to optimize the fluid management in the body cavity to be examined with the endoscope by means of the insufflator or the flushing pump as a function of fluid flows during smoke evacuation or cleaning.

According to a preferred embodiment, the intracorporeal pressure in the body cavity is measured with a pressure sensor and the control unit controls the intracorporeal pressure in an event- and/or time-controlled manner at least during the duration of a cleaning by means of a control of the at least one pump or a control of a pressure regulator such that the intracorporeal pressure does not exceed a predetermined maximum limit value.

In this way, safety can be increased for the person being examined during an endoscopic procedure.

According to a preferred embodiment, the cleaning module is designed to adapt the type of cleaning by changing the cleaning parameters depending on the database classes and/or a degree of contamination, wherein one or more cleaning parameters are selected from a group comprising: type of fluid, fluid volume, fluid volumes, fluid velocity, pressure, pulse duration, number of pulses, pulse-pause ratio and/or total cleaning duration.

For example, cleaning is carried out by means of a cleaning module by applying fluid jets under high pressure to the surface to be cleaned with the aim of at least reducing the substance or vapor to be removed and adhering to the surface of at least one distal end window of the endoscope. Suitable fluids are preferably water or sodium chloride or another physiological fluid. A gaseous fluid is also chosen so that it is physiologically harmless and bio-compatible and can be, for example, carbon dioxide. Parameters such as fluid volume, speed and cleaning time can advantageously be adapted to the degree of contamination determined by the self-learning module.

Limiting the cleaning to a pulse duration of a few milliseconds, for example a maximum of 2000 ms, or specifying a pulse-pause ratio has the advantage that the endoscope operator does not activate the cleaning function for longer than necessary. As a result, not as much fluid accumulates in the body cavity, such as the pneumoperitoneum. By reducing the liquid, less liquid needs to be suctioned out of the body cavity. By limiting the cleaning duration, the cleaning time as well as the cleaning quantity or amount of liquid can be efficiently automated and coordinated. If the self-learning module takes over complete control, such as in a closed neural network, the user no longer has to decide whether and for how long to activate the liquid cleaning function and can concentrate fully on his actual task. This increases the safety of the patient being treated or diagnosed with the endoscopic procedure.

A cleaning parameter change or adjustment can be automated using machine learning or a deep learning model of the self-learning module. For example, parameter adaptation is possible using stochastic approximation algorithms (SA).

According to a preferred embodiment, the self-learning module comprises a cleaning control algorithm based on an image analysis history, which checks the cleaning effectiveness of previous cycles in order to select cleaning parameters adapted to the history, preferably based on stochastic approximation algorithms, in order to achieve the highest probability to obtain an image with sufficiently good image quality.

According to a preferred embodiment, the cleaning parameters are adjusted by means of a deep learning model of the self-learning module or based on a step-by-step increase or by means of maximum settings.

According to a preferred embodiment, for building the reference database, the self-learning module receives training data of image structures of characteristic images of objects to be examined, preferably organs or tissue structures, and/or of smoke via an input interface automatically or by input from a user.

The training data can be received via the cloud, for example. If several hospitals are networked, current data records can be uploaded to the reference database continuously or discontinuously.

According to a preferred embodiment of the method, the following initialization method steps take place before or during the acquisition of image data:
providing at least one further database with technical data of usable image acquisition devices and/or with technical data of cleaning modules;
comparing the provided image acquisition device with the technical data and detecting the provided image acquisition device and, depending on the detected image acquisition device, transmitting stored cleaning parameters to the cleaning module for cleaning activation.

With the help of an additional database, which serves as a parameter reference bank, the user can flexibly choose between different endoscopes and cleaning modules. Thanks to automatic detection, the parameters stored in the parameter database are automatically called up when cleaning is activated.

The method further comprises the following steps:
starting a detection routine to detect structures of a body cavity or a technical cavity, preferably a region or object to be examined, based on the image data, and
continuing the method based on a positive result of the detection routine or terminating the procedure based on a negative result of the detection routine.

If the method is aborted, the connections to the control unit and/or the power supply should be checked in order to then restart the detection routine.

In a preferred embodiment, the method comprises a monitoring routine with the following steps:
after cleaning by means of the cleaning module, analyzing the image data by a control unit.
comparing the image data with stored parameters for a positive cleaning result.
depending on the cleaning result, canceling or repeating the cleaning.

If the cleaning result is not yet sufficient and the optimal image quality is not achieved, another fluid pulse is automatically activated for cleaning and/or drying until the cleaning result is positive or corresponds to the target values. In this way, it can be monitored whether optimal image quality (positive cleaning result) is achieved. If the cleaning process is repeated and the degree of contamination is lower, the cleaning parameters can be adjusted by reducing the cleaning time or the fluid volume. Due to the adjustable cleaning, a smaller amount of flushing gas or fluid is required and the required cleaning flush volume can be reduced to a minimum so that no unnecessary amounts of fluid enter the body cavity.

In a preferred embodiment, the method further comprises the following method steps: Adaptation of the cleaning to the degree of image deterioration, wherein in the event of severe image deterioration, cleaning with liquid is activated and defined cleaning parameters are provided depending on the endoscope provided. Furthermore, if there is little image deterioration or sufficient probability of image improvement after liquid cleaning, cleaning with gas can be activated and, depending on the endoscope provided, defined cleaning parameters can be provided for drying.

If severe contamination is detected, liquid cleaning is preferred, while in case of light contamination or fogging of the optical window, activating gas cleaning or gas drying may be sufficient.

According to a further aspect, a control system is provided for image optimization of at least one image acquired at the distal end of an endoscope, the system comprising an image acquisition device, a control unit with a self-learning module in communication with the image acquisition device for receiving the image data and a memory unit. The self-learning module is designed to determine at least one quality value based on training data by comparing at least one specific image structure with image structures of a reference database stored in the memory unit. Furthermore, the control unit is designed to automatically issue control instructions to a unit of the endoscope or an external unit to activate image optimization based on the determined quality value.

Furthermore, the control system can carry out one or more method steps of the image optimization method described above.

Furthermore, a computer program product is provided which comprises instructions which, when executed by a computer, cause the computer to perform the steps of one of the methods described above.

Furthermore, a computer-readable medium is provided on which said computer program is stored.

Furthermore, a trained model is provided which is trained with training data, and wherein the trained model is designed to perform the steps of at least one of the methods described above.

The training data comprises input data, which is preferably stored in a memory unit and comprises image data, optionally image structures, and/or a collection of features used to train the model. Preferably, the trained model of the self-learning module is trained to classify the image data into the following contamination-dependent database classes or categories:
1. uncontaminated images,
2. contaminated images that can be cleaned by a cleaning module; and
3. contaminated or marked images that can be optimized by modules other than the cleaning module.

With the help of machine-trained database classes or categories, a trained model can reliably and reproducibly classify current image data of an object under investigation, whereby the trained model can detect when a detected image structure has changed, for example due to smoke, condensate or contamination or other disturbing factors. A machine-trained model does not depend on subjective assessments by users. Based on the detected change, control instructions can be issued manually or automatically from a control unit to a unit to activate image optimization. When activated, image optimization can ensure visibility conditions and visual quality during an operation, at least partially automatically.

### BRIEF DESCRIPTION OF DRAWINGS

The disclosure as well as further advantageous embodiments and developments thereof are described and explained in more detail below with reference to the examples shown in the drawings. The drawings are for illustration purposes only and are not to scale. Terms such as above are not to be understood as limiting. The features shown in the following description and the drawings can be used according to the disclosure individually or in groups in any combination.

FIG. 1a is a perspective view of a distal end of an endoscope with a cleaning device according to the disclosure:

FIG. 1b shows an exploded view and a detailed view of FIG. 1a, showing the nozzle and two fluid channels of the cleaning module;

FIG. 2 shows another embodiment of a distal end of an endoscope with a local control unit:

FIG. 3 schematically shows an embodiment with a control unit and units in communication therewith for carrying out the method according to the disclosure;

FIG. 4 schematically shows the data processing:

FIG. 5 shows a flow chart of a cleaning method according to the disclosure with initialization routine:

FIG. 6 shows a flow chart of an automated process sequence with automated analysis steps and cleaning:

FIG. 7 shows a schematic flow chart of an automated method for adjusting the cleaning parameters for a liquid fluid or a gaseous fluid:

FIG. 8 shows a flow chart for schematic representation with an example of a classification in combination with a monitoring routine; and

FIG. 9 shows a schematic flow chart of a method according to the disclosure.

### DETAILED DESCRIPTION OF PREFERRED EMBODIMENTS

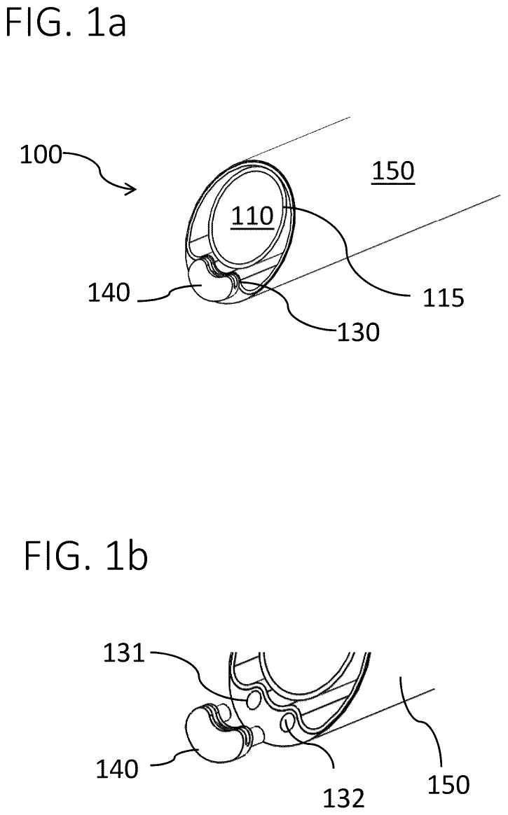

FIG. 1a is a perspective view of a distal end of an endoscope with a distal window 110, with FIG. 1b showing a detailed exploded view of FIG. 1a. The embodiment according to FIGS. 1a and 1b has a cleaning module 130 with a nozzle 140. The optical window 110 of an image acquisition device 115 such as an endoscope is arranged above the cleaning module 130 in a common shaft 150.

The components shown comprise a modular system 100, wherein the cleaning module 130 can be detachably or integrally connected to the other components and/or the image acquisition device.

FIG. 1b shows that the cleaning module 130 has two fluid channels 131, 132. In this way, cleaning fluids such as a liquid and a gas can be transported separately to the optical window 110. The liquid used can be water, saline or other physiological solutions. Air or carbon dioxide are often used as gases. The fluid channels are connected to the respective external sources of the fluids.

To avoid mixing of two different fluids, in this embodiment a two-channel nozzle is advantageously used as nozzle 140, wherein the inlets and the spray channels run separately up to the nozzle outlet. The fluid channels 131, 132 can be activated separately and independently of each other but also simultaneously. Heavy soiling is usually better removed with the help of liquids, while light contamination or condensation on the optical window can be removed using gas. Alternatively, the cleaning module 130 can be designed with a single-channel nozzle in combination with two supply channels (not shown) or with a single-channel nozzle in combination with only one supply channel (not shown), which then intermittently supplies different cleaning fluids.

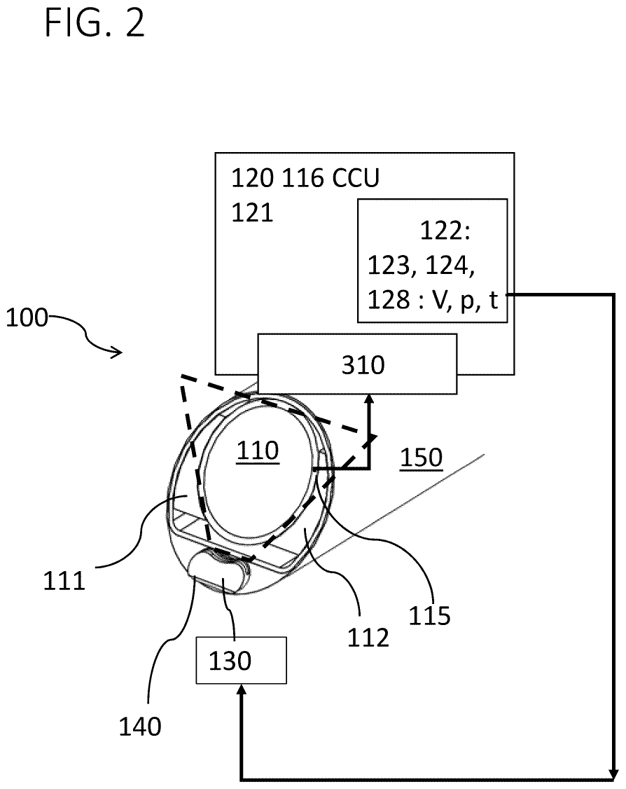

FIG. 2 shows a schematic representation of another embodiment of a system 100 for optimizing an image acquired through an optical window 110 at a distal end of an endoscope by means of an image acquisition device 115. The system includes a control unit 120, which can be provided locally as a camera control unit 116 (CCU).

Furthermore, a memory unit 122 can be provided, which in this embodiment is also arranged locally. Optionally, the control unit 120 and the memory unit 122 may not be arranged locally at the distal end of the endoscope, but may be provided via cable or wirelessly outside the endoscope. The local or external memory unit 122, which can store a plurality of already acquired image data from this image acquisition device 115 (see reference numeral 310 in FIG. 2) or from other image sensors, such as those used in stereo endoscopes (not shown), can be transmitted to the control unit 120 for further processing by at least one processor 121.

The processor 121 may include one or more microprocessors or graphics processors and may be used for image analysis 124 and optics detection 123. The optics detection 123 can be used as an initialization routine to detect the connected image acquisition device 115 and to transmit stored cleaning parameters 128 depending on the detected image acquisition device 115 to the cleaning module 130 for cleaning control or activation. In the memory 122, cleaning parameters 128 such as fluid volume V, pressure p and pulse or cleaning duration t are stored for the cleaning module 130. The cleaning parameters 128 may include further parameters such as number of pulses, pulse duration t, total cleaning duration t, pulse-pause ratio, type of fluid, fluid volume V, fluid volumes (liquid or gas quantity) and/or fluid velocity.

With the help of the cleaning parameters 128, the pressure p and the pulse duration t of a fluid pulse or a plurality of pulses in the cleaning module 130 can preferably be optimally controlled. In particular, very short fluid pulses with a duration in the range of only 200-1000 ms can only be reliably implemented with the help of an automated control system. This allows the system to be controlled much more precisely than through manual activation and deactivation by a user. In FIG. 2, as in FIG. 1, an embodiment of a cleaning module 130 with two separate fluid channels 131, 132 can be used. Other cleaning modules 130, such as those with only one fluid channel 131 that can convey both liquid and gas in one channel to the cleaning nozzle 140, can also be connected to and controlled by the control unit 121 and the system.

The geometry of the nozzle 140 can be configured to be positioned at a predetermined distance relative to the optical window 110 and/or to at least one other window for the illumination devices 111 and 112 such that the fluid jet is directed at least over the entire outer geometry of the one optical window 110 or at least two windows (not shown). This is indicated schematically by the dashed trapezoidal surface. In a further preferred embodiment, the cleaning module 130 and the nozzle 140 are designed so that the side illumination devices 111 and 112 can additionally be efficiently cleaned (not shown).

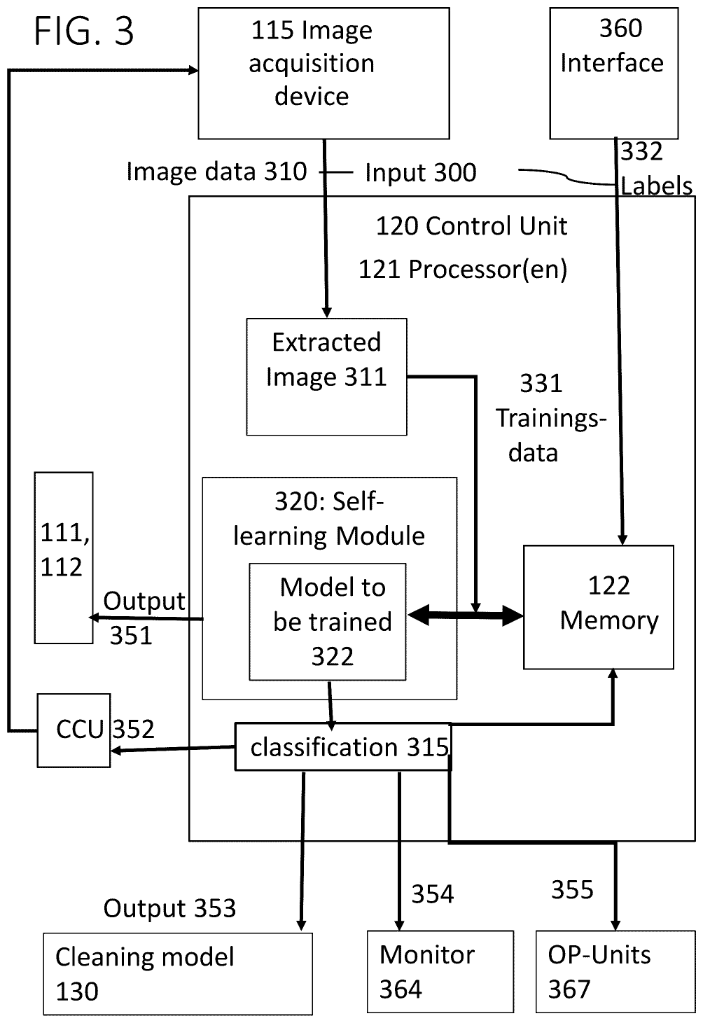

FIG. 3 shows a schematic representation of another embodiment of a system and method for optimizing an image or image data 310, which are acquired by an image acquisition device 115 at a distal end of an endoscope by means of at least one suitable image sensor or at least one camera. Image data 310 of at least one extracted individual image can be input to the control unit via the image acquisition device 115. Further inputs 300 or input signals are possible and can preferably be input via an interface 360 by a user via a suitable interface.

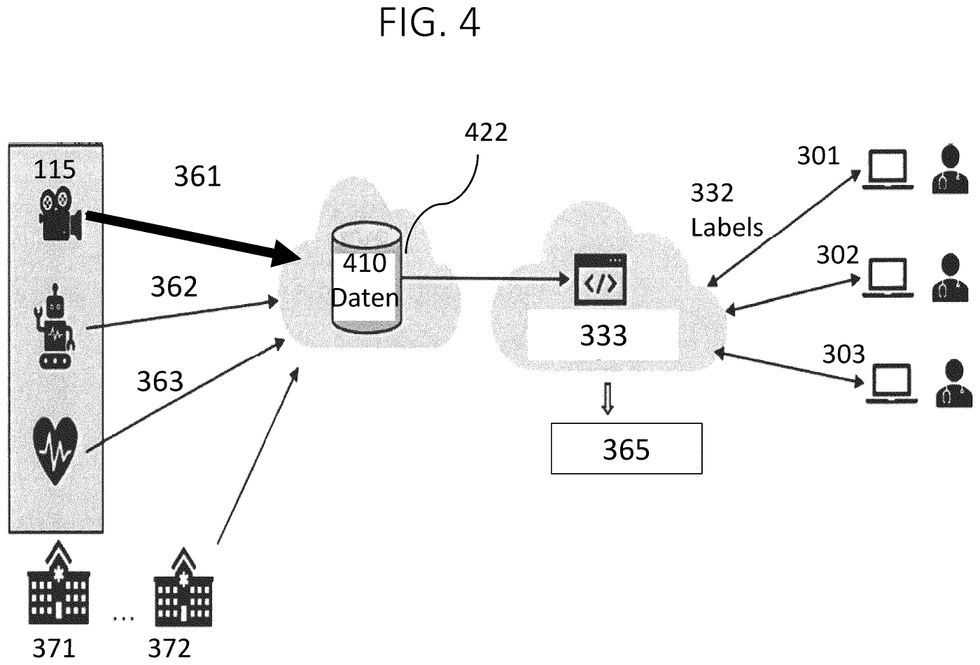

The interface 360 can be implemented in hardware and/or software. In a hardware-based design, the interfaces can, for example, be part of a so-called system ASIC, which contains various functions of the device. However, it is also possible that the interface 360 comprises its own integrated circuits or at least partially consists of discrete components. In a software-based implementation, the interface 360 can be one or more software modules that are present, for example, on a micro-controller alongside other software modules. In order to receive an input 300 or input data, a connection to a cloud can be established, for example (see also FIG. 4).

The central module of the image optimization system is a self-learning module 320. The self-learning module is based in particular on artificial intelligence. In the exemplary embodiment shown, so-called labels 332 are used as input 300. These can be entered via the 360 interface. The labels 332 are preferably understood to be data pairs. The label 322 can be understood, for example, to mean that a known input signal, and thus an input 300, can be assigned to an output signal and a corresponding control instruction or output 351, 353, 354, 355.

In order to train the self-learning module 320 and the model 322 to be trained contained therein, input signals labeled by a user or expert, so-called labels 332, namely inputs 300, as well as known outputs 351, 353, 354, 355, etc., can be specified. In this way, the self-learning module 320 or the model 322 to be trained contained therein teaches itself in such a way that, given an input 300 with labels 332, a control instruction or output 351, 352, 353, 354, 355 is generated to a unit for image optimization based on the data processing. The labels 332 can either be generated by a simulation or provided by experts using an in-house label tool 333, as shown in FIG. 4. The manual generation of the labels 332 can be done using large databases of image data already acquired with endoscopes or using a selected subset of the available data. Furthermore, the labels 332 can also be produced by companies that specialize in the production of such labels 332 and can draw on the corresponding expertise.

As basic input 300 for the self-learning module 320, images or the associated image data 310 of the image acquisition device 115 of an endoscope are provided. In a next step 311, image data 310 from a single image or at least a subset of the image data 310 are extracted from a plurality of consecutive images. This extracted single image, as well as the labeled or unlabeled image data, can be used as training data for the self-learning module 320. The image data history as well as the labeled image data can be stored in the memory 122 (see labels 332 as input and arrow towards memory 122). Based on the combined information from currently acquired image data 310, from which a single image 311 and/or a subset such as image sections are extracted, and the stored training data 331, the model 322 can train by itself.

The information of the image 310 extracted in step 311 is preferably pre-processed. Pre-processed information refers to specific information about the current imaging. The image data can be checked according to different criteria and, for example, object detection can be carried out. These are, for example, detected image structures, such as structures of tissues or organs, which were acquired by the image acquisition device 115. However, information such as brightness or other environmental conditions can also be obtained from the individual image. The information obtained can be stored temporarily or long-term in the memory 122.

The trained model 322 is designed and trained to evaluate the extracted individual images 311 or individual image sections or pixels, on whether contamination is present and how severe this contamination is. In order to quantify the degree of contamination, a classification 315 into appropriate categories is carried out. The classification 315 contains at least two classes. For example, the two classes could include insufficient brightness and sufficient brightness. If the brightness is sufficient, there is no output to activate an image optimization unit. If the brightness is insufficient, at least an output signal or a control instruction can be issued to optimize the image. For example, the output 352 is transmitted to the image acquisition device 115 in order to control the camera control unit (CCU) accordingly. Alternatively or additionally, the monitor 364 can be adjusted via an appropriate control signal. The brightness can be improved via control instructions to at least one illumination device 111, 112 by outputting an output signal 351 to the respective illumination unit 111, 112.

A variety of classifications are possible. For example, the categories “sharp” and “fuzzy” can be distinguished, and if a blurred image is determined via an output 352, a focus system can be activated by the camera control unit. Additional units such as operating units 367 such as an insufflator or other units located in the operating room that can be used advantageously for image optimization can be controlled via output 355.

A common cause of image disturbance or deterioration is the presence of contamination, which can be caused by blood or fat or other contamination factors. In the classification step 315, the trained model 322 can determine the degree of contamination and output it as an activation signal in the form of the output 353 to the cleaning module 130. Depending on the level of contamination, a liquid pulse or a gas pulse can be activated. In this way, an automated, self-learning module 320 can learn to detect dirt and activate the trained model 322 of the cleaning module 130 with a specifically aligned nozzle 140 (see e.g. FIG. 2) so that not only the optical window 110 of the image acquisition device 115 but also the illumination devices 111 and 112 are cleaned. If at least one illumination device 111, 112 is cleaned, the service life and the light output can advantageously be increased at the same time.

Cleaning can be done automatically by the trained Model 322 so that the user is not disturbed during his examination or work. The cleaning time is optimized so that the user's vision is not impaired or is only impaired for a very short time. This is preferably done by setting high pressures, which can be set briefly, e.g. for a maximum duration of 1000 ms. Pressures of up to 3 bar ensure efficient cleaning, wherein, as with a windscreen wiper, the impairment of visibility can be set to be very short time and therefore barely noticeable.

At the beginning of the training, an expert or the user can also manually enable cleaning or check the dirt detection routines. The digital image analysis using the trained model 322 can be used to trigger the cleaning semi- or fully automatically. The longer the Model 322 is trained, the more reliable the classification results are and the cleaning function no longer needs to be released manually.

FIG. 4 shows schematically an embodiment of a data processing. The database 422, which in this example is centralized and can be provided in the form of a cloud, stores the raw data 410 for the process. The input data is shown by arrows to the centralized database 422. In particular, camera data 361 can be imported. This camera data 361 or image data 310 may be provided by a plurality of hospital networks 371 and 372. Alternatively, only a single camera module or image acquisition device 115 can provide the raw data 410.

Furthermore, automatic additional data 362 can be imported. These data 362 can contain additional information from monitoring systems or robot systems used. Such additional data 362 can be related to the operating room and can indicate the temperature of the operating room or brightness of the operating room. Furthermore, patient data 363 such as vital data but also user-specific data can be made available for the raw database 410. All data can be stored centrally. Alternatively or additionally, decentralized storage is also possible. The raw data 410 of the reference database 422 can be accessed by another unit of the network, such as a labeling unit 333. In this embodiment, this labeling unit 333 is a cloud or a web service that is suitable for labeling or marking the input information.

The raw data 410 may be surgical videos without labeling and may be stored in the centralized reference database 422. An internal team or external experts can then label the raw data 422 using the web labeling tool or unit 333. In this way, the labeled surgical images or other suitable data sets can, for example, determine image quality disturbances and quality values.

The labeling can be carried out by different persons 301 302 303. These can either be experts or users or externally commissioned labeling teams who can assess the quality of the surgical images and can draw on the necessary expert knowledge. The generated label 332 can be used for training data 331 (see FIG. 3) for the self-learning module 320 and thus for neural networks. The training data 331 or labeled data sets 365 are the input data for the trained model 322 (not shown in FIG. 4). The labeling process can be used to determine a quality value that can be used for later comparison with a specific image structure in a reference database.

Basically, the learning process in the self-learning module 320 is based on at least one model 322, which in a preferred embodiment is a stochastic model and is based on probability considerations as to whether the image quality can be optimized. In other words, the current image is compared with database classes and it is decided, preferably with the help of labeled data sets 365, whether the image can be optimized. Decisions are made based on appropriate marginal probabilities and a new database class may also be required and added by the self-learning module 320.

In the construction and training phase of the model 322, an open AI network based on artificial intelligence can be used. Here the image data sets can be corrected by experts 301, 302, 303. These experts can compare or evaluate existing image databases with the latest results. In this way, an existing data set is corrected and, in addition, the reference database can be expanded to include new data sets. The labeled data sets 365 can thus ensure the quality of the classification process.

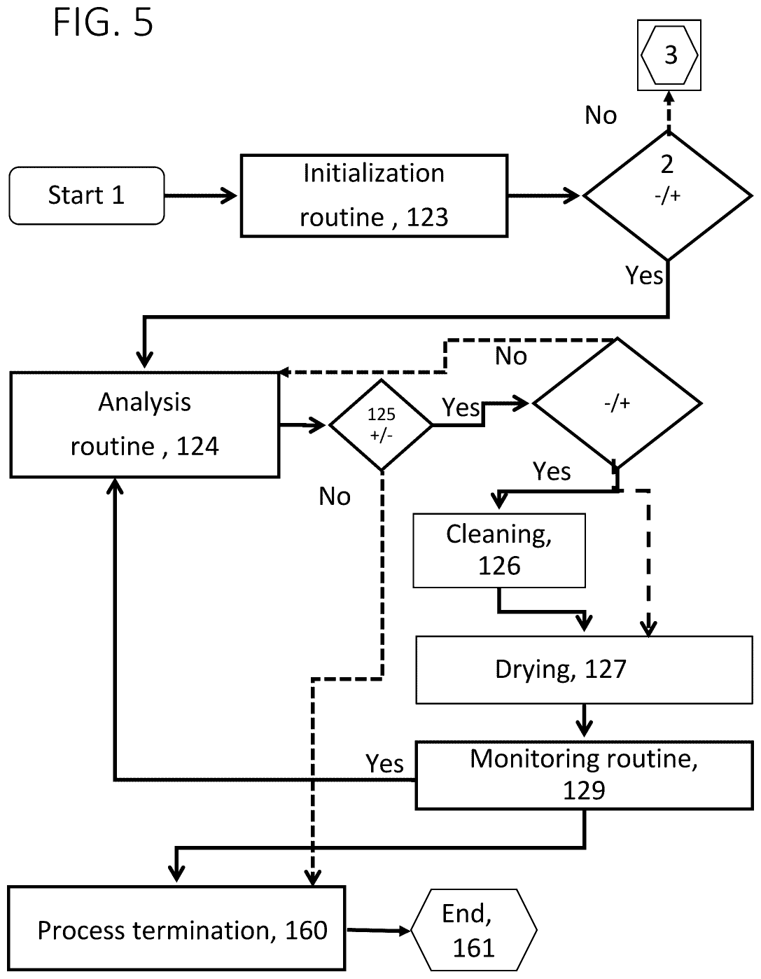

FIG. 5 shows schematically the method steps of an embodiment of the method according to the disclosure for image optimization. The procedure begins with step 1 (start) and starts a suitable software or computer program through the control unit to start the image acquisition. The initialization routine takes place in step 123. This initialization routine starts an image detection and/or an optics detection. In this way, the software detects which optics or camera sensor is connected. The connected illumination devices can also be detected by the initialization routine. With the help of the initialization routine, the optics detection can also determine which type of endoscope is being used and which cleaning modules are connected.

In decision step 2, for example, it is checked whether a video signal is present or whether an optic is connected. In case no optics are detected in step 2, process interruption 3 is initiated (indicated by the arrow NO in the flow chart). This process interruption 3 can be reported to the user via a monitor or an alarm signal so that the user can take appropriate steps to correct the problem. For example, the connections to the control unit or the power supply can be checked.

In case a positive result is detected in the optics detection, the flowchart follows the YES arrow to analysis routine 124. The analysis routine uses a self-learning module 320 (see FIG. 3) and applies artificial intelligence. The currently acquired image data is compared with already saved image data and a quality value can be determined based on this comparison. This comparison can also be used to automatically detect the operating room environment or contamination of the field of view.

In the following step 125, a query is made as to whether the acquired image has detected an object or not. The object detection can, for example, detect a technical cavity to be examined, a body cavity to be examined, tissue structures or an operating room environment. If this query 125 is negative (NO for the dashed arrow), the process is aborted in step 160. Without object detection, the contamination or other disturbance of the image quality cannot be detected and the method can be terminated in step 161.

If the query in step 125 is evaluated positively, for example if contamination or a blurred or too dark image is detected, the YES step arrow leads to a further decision query. A distinction is now made as to whether there is a contamination or malfunction that can be remedied with the cleaning module or whether there are other disruptive factors that cannot be remedied with the cleaning module. If other interference factors are present, the analysis routine 124 is carried out again (see dashed NO step arrow back to 124).

If the result is positive, i.e. if there is a contamination that can be eliminated by means of a cleaning process, the decision tree is now such that either a cleaning 126 with a liquid fluid or a cleaning 127 with a gas or both are carried out one after the other. After a liquid cleaning 126, the gas cleaning 127 can be used for drying. Exclusive drying 127 can be advantageous, for example, if condensate has formed on the optical window. A gas supply in step 127 can dry the condensate and correct the image disturbance. In addition, combinations of simultaneous activation of cleaning with liquid (step 126) and cleaning with gas (step 127) not shown here are also possible.

Advantageously, the cleaning by liquid 126 is only activated for a short period of time of a few milliseconds so that the user is not disturbed during operation or examination. This so-called windshield wiper effect can only be carried out by software control, since such short periods cannot be adjusted manually.

After cleaning is completed, monitoring routine 129 is started. Cleaning can be carried out depending on the degree of contamination and can be optimized by the monitoring routine 129. Typically, heavy contamination is cleaned with liquid (step 126). In case of slight contamination or fogging of the optical window, cleaning with gas and thus drying 127 is sufficient. The cleaning steps are activated either automatically or after approval by the user.

After activation, the monitoring routine 129 checks whether the field of view and the image quality are optimal again. If this is the case, it goes back to analysis routine 124 (see arrow YES). A closed control loop is provided for the monitoring routine 129, which checks whether or not predetermined target values of the image quality are achieved by the activated cleaning 126 or drying 127. Monitoring routine 129 can increase patient safety. If the cleaning result is not optimal, a repetition of the cleaning 126 or drying 127 can be initiated. If the monitoring routine 129 determines that no improvement in the quality of the image is achieved by cleaning 126 or 127, the optimization process can be aborted by means of the cleaning module in step 160 (see end 161).

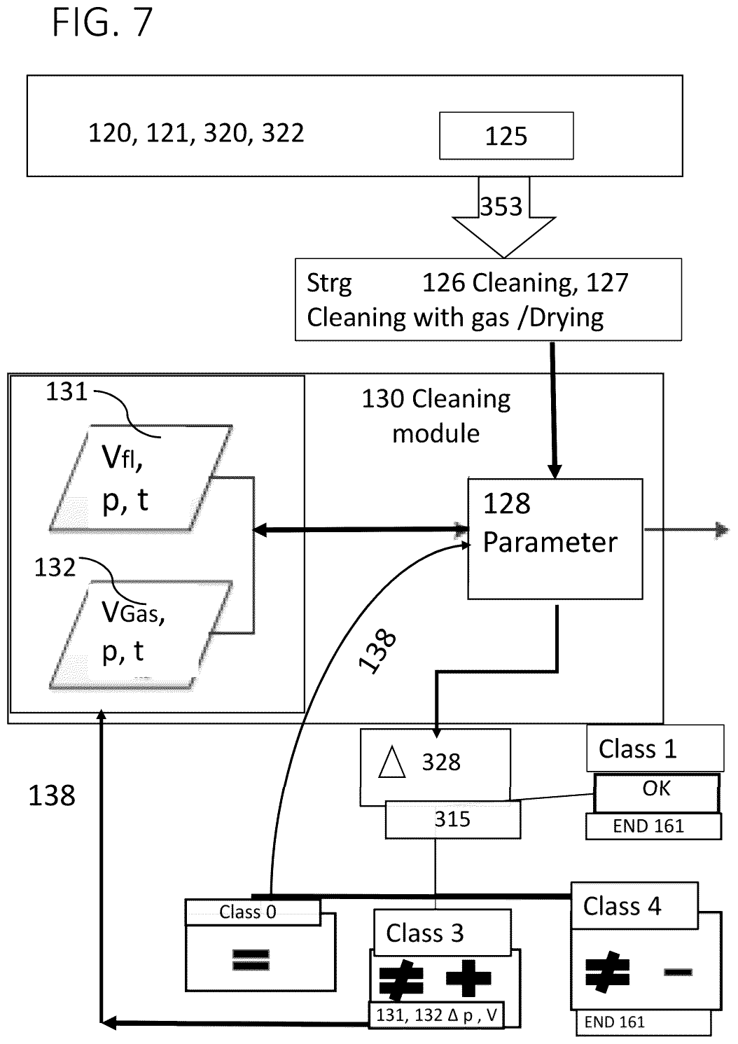

Based on the self-learning module 320 and the trained model 322 contained therein (see e.g. FIG. 3), not only automated analysis routines 124 and subsequent automated cleaning processes 126, 127 can run, but also the cleaning parameters can be adjusted. This is explained in more detail in FIG. 7.

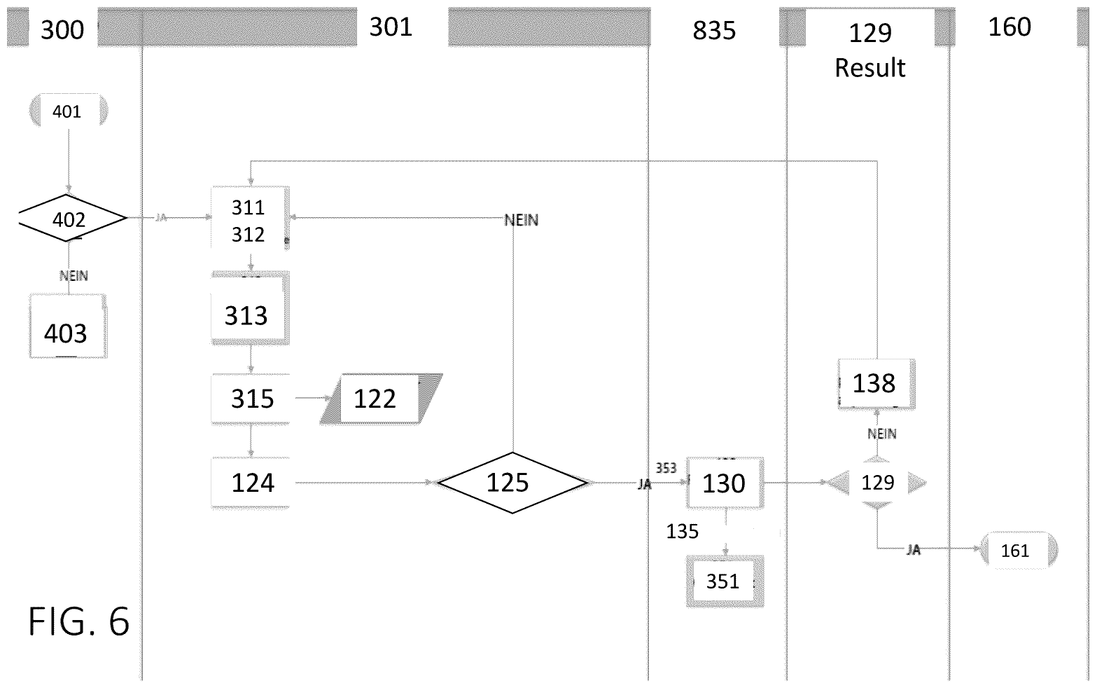

FIG. 6 shows a further embodiment of the method according to the disclosure with a schematic flow diagram. The flow chart is divided into five areas, indicated by columns: the first area of the input 300, the second area of the analysis 301, the third area of the process execution for optimization preferably by cleaning and the fourth area of the optimization result with monitoring routine 129 and parameter adjustment 138. Finally, the possible termination of the process 160.

In the area of input 300, the method is started in step 401. A video signal from an endoscope can be provided as an input signal. In step 402 it is checked whether a video signal is present. Based on this check, either a positive result (YES) or a negative result (NO) is obtained. If this query 402 results in NO, the process is aborted in step 403. Since there is no video signal in the case of process termination 403, it should be checked after process termination 403 whether all connections exist and whether a power supply is available.

If the check 402 produces a positive result (YES), the process continues in the analysis area 301. In step 311, the so-called extraction of at least one image takes place. This means that individual images are extracted from the video signal. In a step 312, the images can optionally be divided into image sections. In this way, the image can be divided into different sections, e.g. A, B, C, where e.g. C can represent a central region of the image.

In the next method step 313, a preliminary analysis of the images takes place or, if step 312 has been applied, a preliminary analysis of the image sections. In the pre-analysis step 313, the images are pre-processed and compared with a database stored in the memory 122. Preferably, a comparison of at least one specific image structure with image structures of a reference database takes place. Different criteria can be checked and object detection or actual data collection can take place.

In the next step, classification 315 into different classes or categories can take place. The image classification is based on a quality value that can be determined using a training-based model (see reference number 322 e.g. in FIG. 3). This model 322 is part of a self-learning module 320 in a control unit 120 of the image optimizing system and cooperates with the reference database contained in the memory 122. This memory unit 122 can store the history of previous image acquisitions. Labeled video images (see 365 in FIG. 4) of an endoscope can also be stored in the memory 122 in order to determine the quality value. In this way, an acquired image can be qualified in terms of image quality using stored parameters or characteristics.

The classification history is stored in the memory 122 and is continuously updated with the currently acquired image data. Not only the acquired images from the previous steps but also the corresponding evaluations of the video signals are saved. memory in memory unit 122 can be local or decentralized. Through the so-called image history, continuous documentation can be carried out and the model 322 to be trained (see e.g. FIG. 3) can be continuously optimized. The larger the retrievable data sets, the more precise the machine learning can be and thus the learning ability of the model 322 can be increased. With a sufficiently large data set, for example, artificial intelligence with a deep data model can be used.

In the classification step 315, individual image sections or pixels can be evaluated as to whether contamination is present and how severe the contamination is. This makes use of the existing database classes or categories. For example, a class 1 “OK”, i.e. “optimal images”, and a class 2, i.e. “contaminated images”, can be distinguished. Other classes such as well-lit images and poorly lit images can also be stored in the database class. Additional information about, for example, smoke or brightness or focus settings can be used for the following method steps and control instructions or output 353 or activation signals 135 to optimize the image. For example, if smoke is detected, communication can be established with at least one other operating room unit 351 to extract the smoke.

The analysis 124 evaluates the type of contamination based on the pixels and optionally based on the position in the image. If contamination is detected in class 2 “contaminated images”, a further distinction can be made as to whether this contamination can be cleaned or not. In a preferred embodiment, in analysis step 124, for example, the degree of contamination can be determined based on an evaluation of the image history stored in memory 122. In step 124, a total evaluation of the image quality is carried out, depending on the percentage of “contamination” allowed. Image edge areas can be given less weight than the image center.

In step 125 it is determined whether an object has been detected or not. In addition, once an object or image structure has been detected, it can be determined whether there is contamination or not. The type and degree of contamination was already determined in advance by a suitable classification 315 with the aid of a comparison in the classification step 315 with the reference database using algorithms in automated manner in the self-learning module 322 of the control unit 120 (see FIG. 4). In step 125, not only can possible contamination be checked, but other disturbing factors can also be excluded, e.g. the exclusion of smoke or other conditions such as insufficient contrast, sharpness or light.

If it is determined in step 125 that the quality value is already optimal, an optimization, for example by a process implementation 835 such as cleaning, is not necessary. Since the cycle for optimal image quality is completed here, the method returns to one of the steps 311 or 312. A lack of contamination is schematically indicated by the arrow NO, which leads from step 125 to steps 311 or 312. A restart at 311 can occur continuously or at set intervals.

If in step 125, on the basis of the preceding analysis 124, the image quality was assessed such that cleaning or optimization can be carried out by a process execution 835, the method continues (see arrow YES in FIG. 6) and an activation signal is output via an output 353. In the area of process implementation 835, for example, a cleaning module 130 can be activated to carry out cleaning 126 or drying 127 using a fluid. The process execution 835 of the cleaning process can be activated automatically by the self-learning module 320 or, alternatively, can be activated manually after the self-learning module 320 has recommended cleaning. Cleaning can be carried out via a nozzle with liquid 126 or with gas 127 to initiate cleaning or drying. These two cleaning types 126 and 127 (not shown in FIG. 6, see e.g. FIG. 5 or 7) can also be combined.

In a preferred embodiment, in the case of larger contaminations, the fluid is liquid and the cleaning can be carried out with at least one fluid pulse with a duration of only a few milliseconds up to a maximum of a thousand milliseconds. If, in addition or as an alternative, the presence of smoke is detected, for example, communication 351 with another device unit can be established by means of an activation signal 135. Another device unit may be an operating room device unit 367 (see FIG. 3), such as a smoke evacuator, which is activated when smoke has been detected by the previous steps. Furthermore, an insufflator pump or other operating units 367 can be controlled in order to obtain an optimal cleaning result or the desired image optimization.

In the next area, the result of the optimization is monitored 129. If the activated process execution 835 was a cleaning, the cleaning result is first compared with a database in this area. This is done in method step 129, which includes a monitoring routine 129. With the help of the monitoring routine 129, the cleaning effectiveness of the previous cycles can be checked by evaluating the image history from the memory 122. In an open artificial intelligence network, the comparison with the reference databases can be corrected by an expert if necessary. The cleaning results or optimization results can be assessed by a team of experts or a single expert, thus checking the algorithms used and the trained model. In a closed neural network, corrections can be made automatically by the self-learning module 320, while in an open neural network, experts can also decide whether a new database class is required. If necessary, an expert can not only make corrections but also carry out preliminary control. Alternatively, after a sufficient learning phase of Model 322, a model-based feedforward control can also be set so that, for example, definable set point values for the pressure in the body cavity are not exceeded.

In the case of an open neural network or a closed neural network or a hybrid form of both (“semi-supervised”), process optimization and/or parameter adjustment 138 shall be carried out before the restarting of the procedure (repetition of steps 311-315, 124-125). This allows a model to be trained to be optimized during the training phase in order to achieve the desired optimal results. The cleaning parameters can be adjusted, for example, by gradually increasing the fluid volumes in step 138.

According to an exemplary embodiment of the cleaning adaptation 138, the self-learning module 320 can shorten the duration of the cleaning by checking the cleaning result and adjust the cleaning parameters in step 138 so that the cleaning duration is, for example, only between 200 ms and 800 ms. The cleaning duration can be shortened depending on the degree of contamination and by simultaneously increasing the pressure. The pressure or the amount of fluid during cleaning 126 must be set sufficiently so that the contamination can be removed efficiently. In the self-learning module 320, the pressure curves are automatically optimized by changing parameters in step 138 using the trained model 322. The criterion of minimal fluid consumption must also be met.

Preferably, the following criteria are taken into account for parameter assignment and parameter optimization:

The main criterion is cleanliness and complete removal of contamination.

A secondary criterion is the minimum cleaning time and minimum fluid consumption.

The parameter adjustment 138 is preferably carried out in such a way that the main criteria and preferably also the secondary criteria are met with a high degree of probability. The aim of the model 322 to be trained is therefore to optimally remove dirt while keeping the cleaning time as short as possible in order to cause minimal disruption to the user.

Basically, the process is based on probability considerations as to whether the image quality can be optimized. The above criteria should be met with a high degree of probability. The procedure for image optimization by changing parameters (height, order, etc.) can be different for different image classes or degrees of contamination and can be stored in a data set in a parameter database in the memory 122. The self-learning module 320 is configured so that initial parameter setting and parameter adjustment 138 can be carried out automatically. The cleaning parameters can be assigned and adjusted using image analysis 124. Based on optimal parameter settings and adaptive and simultaneously predictive control by the self-learning module, the highest probability of obtaining an uncontaminated image in sufficiently good image quality for the user can be achieved.

In a preferred embodiment, for example, the degree of contamination can be determined compared to the image history. The degree of contamination may no longer be present, may be less, may have remained the same, or may have worsened. Depending on the degree of contamination, a gradual increase can be made, for example by increasing the duration of the cleaning pulse or the pressure used. On the other hand, in case of high levels of contamination, maximum settings can be used to optimize the image. These possible parameter adjustments are marked in the flow chart by the reference numeral 138. The adjusted parameters can be output to the cleaning module.

If the cleaning result is better but still not satisfactory, the cleaning can be repeated with different parameters. After the cleaning parameter adjustment 138, the method is continued back in the area of analysis 301 to the step 311 of extracting the image sections. This is followed by classification 315 and further analysis steps, which culminate in contamination detection in step 125 to decide whether a positive change can be made by cleaning or not. After activation of the cleaning module 130, the cleaning result is checked again in step 129. If the result is very good, the cleaning process can be stopped. This is marked with the YES arrow to the end 161 in the process termination area 160. On the other hand, the analysis steps in area 301 can be repeated again.

FIG. 7 shows schematically in a flow chart in particular an automated method for adjusting parameters, which can be used for example in the method step 138 of FIG. 6 or can be carried out by means of the system shown in FIG. 3 comprising a control unit 120 and one or more processors 121.

In this embodiment, cleaning parameters 128 for a dual-channel cleaning module 130 designed for cleaning with both a liquid fluid and a gaseous fluid are adjusted to perform optimal cleaning 126, 127. As an alternative to a two-channel cleaning module 130, a three-channel cleaning module 130 or two cleaning modules 130 can also be provided in order to optimally clean two distal windows of a stereo endoscope.

At the beginning of the method, the cleaning parameters are selected or adjusted depending on the cleaning module 130 used. Essential parameters are the liquid quantity V_{fl}, such as a quantity of water or physiological sodium chloride solution from a liquid channel 131, an optimal pressure p and a time parameter t such as the cleaning duration t or intervals. Other parameters not shown in FIG. 7 such as pulse-pause ratios, number of pulses, etc. are also adjustable. With the second fluid channel 132, gas can be used for cleaning or drying, wherein a gas quantity V_{gas}, the pressure p and the duration t of the gas pulse are shown as examples.

In the control unit 120, the self-learning module 320 is designed to detect contamination and preferably a degree of contamination. If contamination is detected in step 125, a corresponding output signal 353 can be output to a cleaning module 130. A control instruction “Ctrl” activates a cleaning with a liquid 126 and/or gas 127 via an output 353.

Depending on the degree of contamination determined, the parameters 128 for cleaning can be adjusted. The adjustment of parameters 138 can also be made after determining the cleaning result. If the trained model 322 is based on an open neural net, the optimization can be carried out with the help of a classification 315 in a loop. This is shown schematically with a tree diagram and arrows and described below.

In step 328, a query is first made as to whether or not the image shows a change after cleaning. The cleaning results are determined using the self-learning module 320. The cleaning result can be divided into four classes depending on a change:
Class 1 includes a good result i.e. “OK” after cleaning by liquid or gas, so that here the process can be stopped in step 161.
Class 0, corresponds to a cleaning result without any change and is marked by the equal sign “=” i.e. the image has remained the same.
Class 3 corresponds to a positive cleaning result, i.e. a change (marked by the unequal sign “#”) for the better “+” in other words an optimisation of the image was made.
Class 4 corresponds to a cleaning result with a change “#”, whereby the image is worse “−” than before.

If Class 1, Class 3 with a sufficient image optimization or Class 4 result, the cleaning process in Step 161 “End” is interrupted because no further cleaning is necessary or no success can be achieved by the cleaning. In the latter case, other optimization methods can be determined based on pixel evaluations and parameters such as color, resolution, light, contrast and/or sharpness can then be adjusted.

A parameter adjustment 138 is carried out during classification into class 0 and class 3 if a positive change is detected which is not yet sufficient. Since cleaning and adjustment of the parameters in the cleaning module is necessary here, a parameter adjustment 138 is carried out in the loop, wherein parameters 128 of the cleaning module 130 are adjusted for liquid or gas by changing the fluid quantity or the pressure or the pulse duration t.

If the cleaning result has been classified into a category where an adjustment of the parameters takes place, it is again determined in step 328 whether there is a positive, negative or zero change. If the cleaning result according to classification 315 is better but still not satisfactory, the cleanings can be repeated with different parameters. Then the cleaning result is checked again in step 328. Finally, if the result is class 1 “OK” or according to class 0 “=” constant, the process of cleaning can be interrupted in step 161 “END”.

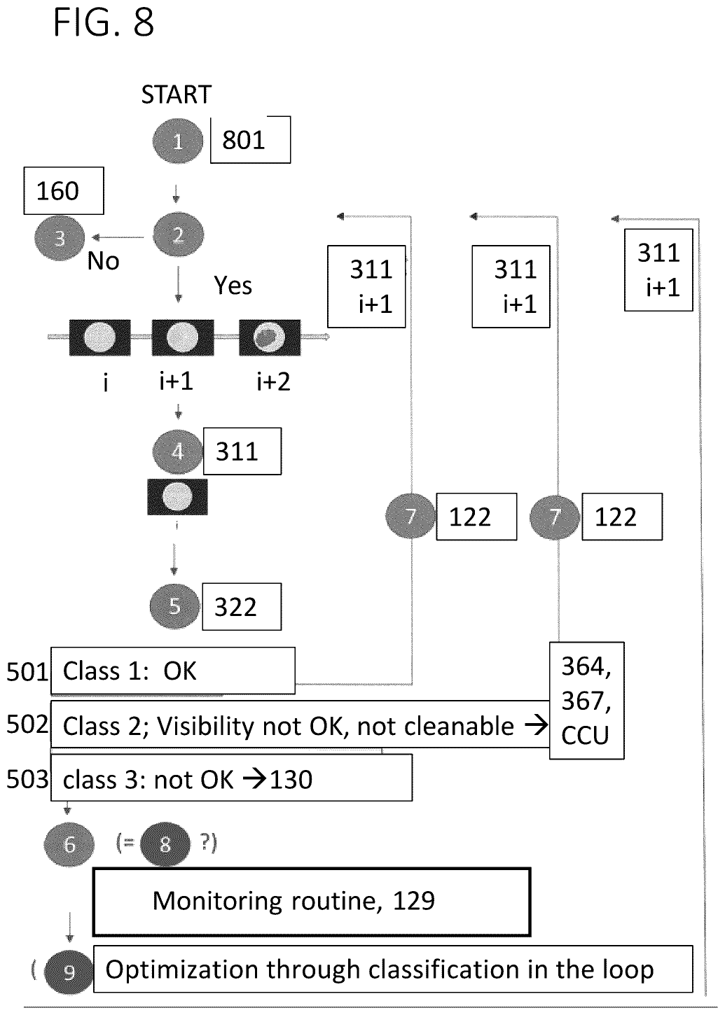

FIG. 8 shows a flow chart for schematically illustrating, for example, another embodiment comprising an application of a trained model 322 (step 5) and a monitoring routine 129. In FIG. 8, as in one of the previous methods, the video signal is first started in step 1. The process begins at step 1 and the subsequent step 2 checks whether a video signal is actually present. If this is not the case, it goes to the process stop 160 according to the arrow “NO”. Here one can check that the connections are correct and then start again from the beginning with a restart at step 801.

If the query in step 2 is positive and answered “YES”. At least one image is acquired. In FIG. 8, a plurality of images i, i+1 and i+2 are shown schematically. In the next step of pre-analysis, a single image is extracted, in the case shown, image i is extracted (step 4). This corresponds to the general method step of extracting an image 311. The trained model can then be applied to the extracted image in step 5. In step 5, the classification is carried out which includes at least three classes (see 501, 502, 503).

In the shown exemplary embodiment of the optimization method, the acquired image i is classified into three different classes.

In classification step 501, class 1 is determined, wherein the image is recognized as not contaminated and is thus classified as “OK”.

Step 502 concerns class 2, where visibility is not OK and there is contamination or other impairment. This category is classified as “non-cleanable” because in Class 2 the visual impairment or contamination cannot be remedied by cleaning with liquid and/or gas. This can be the case, for example, if the focus setting is incorrect. In this case, the camera's control unit can be controlled, for example, to adjust the focus to optimize the image. This is shown schematically with the arrow pointing to the CCU (Camera Control Unit). Alternatively or additionally, depending on the disturbance, other device units, such as a monitor 364 or an operating room device unit 367, such as an insufflator, can be controlled. For example, if a blurred image is detected in classification step 502, the camera focus can be adjusted by the CCU so that a sharp image of the object to be examined is created.

All information acquired by the self-learning module 320 during the application of the model 322 (step 5) and thus e.g. in the classification steps 501 or 502 can be stored in the memory in the memory unit 122 in step 7. Then, the next image i+1 is extracted in step 311 and the analysis method starts again at step 2.

In addition, the acquired image can be assigned to a class 3 after the analysis steps in step 503. Class 3 means that the image is contaminated and can be cleaned by a cleaning system or cleaning module 130 (this is shown here schematically with a “not OK” and an arrow to 130 i.e. the cleaning module). In step 6, the classified images can now be further evaluated and analyzed. This may involve checking, prioritizing and/or evaluating the contamination. Optionally, the image can be divided into sections in order to locate the respective contamination.

With the help of the trained model 322, tissue structures, objects, organs or other objects to be examined can be detected and distinguished from dirt or smoke. More classes than those listed above can be added, either by an expert or by the self-learning module.

After a preliminary analysis and classification 5 by the trained model 322, a further analysis can be carried out in step 8. Here, an evaluation of the image history can take place and the presence and type of contamination can be determined. Many categories are conceivable here, not just those listed above. The level of contamination can be further graduated and categories such as low contamination “x” more contaminated “xx” or very heavily contaminated can be specified. Other categories may also be: contaminated with blood, fat or other disruptive factors such as condensate or smoke.

Furthermore, further analysis can also be used to weight the image areas, which the trained model can determine itself. Each image area can be assigned a permissible percentage of contamination. A preferred embodiment of a self-learning method consists in that a higher priority is assigned to the image center and better quality values are required here than in outer areas.

In method step 8, a decision is made as to whether cleaning is necessary or not. If no cleaning or optimization is necessary, the cycle is aborted and terminated for optimal image quality. The method can be repeated continuously at step 2 or restarted at specified intervals by restarting 801. When using the trained module comprising an open network, an additional step for continuous process optimization, carried out with the help of experts, is preferably carried out before the restart 801. In this way, the process can be continuously optimized.

If it is decided in step 8 that cleaning should be carried out, the cleaning module 130 is activated via an activation signal or output signal 353. After activation of the cleaning step, a comparison with the database can be made by means of a monitoring routine 129.

All results can be stored in a memory 122 in step 7. If a contaminated image is determined as a result after step 6 or step 8, the cleaning parameters are preferably adjusted using the model 322 based on an open neural network with the aid of classification steps and an optimization 9 in a loop. This principle has already been described schematically and in more detail in FIG. 7 in connection with a parameter adjustment 138.

After completion of the above-mentioned method step 9, a new image i+1 is extracted and a new cycle of analysis steps 2 can begin at step. This is indicated by the upward arrows to step 2. Then the process starts again and the image optimization can begin again.

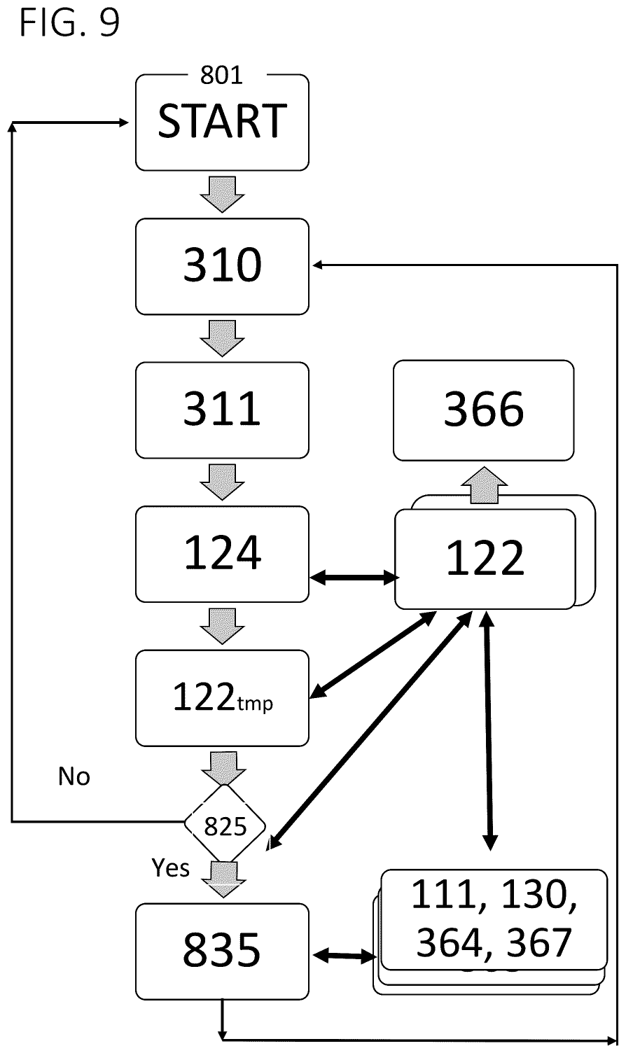

FIG. 9 shows a further schematic flow diagram of the method for image optimization according to the disclosure. The process is started at the point marked with START or 801. If a video signal is present, data acquisition can begin in step 310. In step 311, images are extracted. After extraction 311, individual images are passed on, which can also be broken down into image sections. Extraction 311 is followed by data preparation, where the actual data set is determined and pre-processing of the images can be carried out.

In the subsequent step 124, the data are evaluated by means of data analysis. This data analysis 124 is carried out with the help of a self-learning module 320 and a reference database which is stored in the memory 122. The self-learning module is designed to determine a quality value by comparing at least one specific image structure with image structures in a reference database. The interaction with the reference database during the quality value determination is indicated by the double arrow between the data analysis 124 and the memory unit 122. The found actual state can be stored in a temporary database “122tmp”. If the result is that optimization is not possible or cleaning is not necessary, this temporary image can be deleted later. However, if contaminated or otherwise impaired images are detected by the analysis, it is advantageous for these data to be stored as training data for a model 322 of the self-learning module 320 in the reference database in the memory 122.

The reference database in the memory unit 122 consists of a plurality of databases such as image parameter data sets based on an image database and a parameter database such as cleaning parameters or monitor parameters. In the reference databases of the memory unit 122, good or contaminated images can be classified based on image section data, pixel evaluation, contrast, color, sharpness, resolution, brightness, detection of smoke and/or objects.

In addition, the storage of additional information or additional evaluations 366 is advantageous. A possible additional information 366 is, for example, an acquired image with smoke. In addition, a variety of objects to be examined can be stored as additional information: tissue, veins, tendons or bones. Additional information can also concern the illumination condition and refer, for example, to certain wavelengths of light. Any information is suitable as additional information if the goal of optimizing the image quality of the images acquired by the endoscope unit is met. Optimization can be achieved not only through a cleaning process, but also through other optimization processes, such as changing the settings for playback on a monitor such as an LCD display.

For example, the structures to be examined in the field of view can be identified better or in more detail when certain fluorescence imaging techniques are used. For example, the use of indocyanine green (ICG) can make anatomical structures more visible using light with near-infrared (NIR) wavelengths. For this purpose, the light can be optimally adjusted to the corresponding wavelength by means of the control unit 120. In addition, 3D technologies can also be used and optimized parameters can be adjusted. If, for example, fluorescence technology is used, the wavelength range of the illumination device can be adjusted accordingly. For the fluorescence technology, corresponding video signals are then also stored and a database of additional information 366 is made available in the memory 122 for this special technology. This and other additional information may be used to enhance the image depending on the technologies used.

Further additional information may contain parameters to counteract color distortion in the image reproduction of a monitor. One criterion for good color-accurate image reproduction on the monitor is, for example, correctly set color spaces. Suitable parameters here are brightness, color saturation settings or focus settings. Since the image quality can also be modulated by the display or monitor, it is important to save the optimal settings and adjust them depending on whether changes in the basic conditions have occurred. A monitor can also be customized to suit the user. For example, if the user wears glasses or has red-green color blindness, user-specific settings can be saved as additional information and activated when the user is detected by stored data (see reference numeral 363).

The additional information can also relate to an endoscope unit comprising an electronic image acquisition device such as CCD or CMOS systems or at least two image acquisition devices for stereo systems, which are arranged at the tip of the endoscope. The decisive parameters here are optimal light intensity, depth of field and contrast, as well as magnification options if necessary. The main task is to prevent or eliminate disturbances such as contamination or condensation and to reproduce the detected surfaces or structures of internal organs with the highest image fidelity.

Based on the reference databases stored in the memory 122, the trained model 322 can now decide in step 825 whether an optimization of the acquired image can be carried out or not. If the image cannot be optimized, it goes back to START at step 801 and the process starts again from the beginning.

If optimization is possible, the decision path does not go along the NO arrow, but along the YES arrow to activate the next method step 835 of process execution. To activate image optimization, at least one device unit from a plurality of device units, such as the cleaning module 130, a monitor 364 or an operating device 367, is available. The plurality of device units are in communication with both the memory unit 122 and the control unit 120 (not shown here). Control instructions are transmitted to at least one of the device units depending on a quality value determined in the analysis in order to finally carry out the optimization in step 835.

An example of optimization is the activation of a surgical device 367 such as an insufflator or the activation of at least one illumination device 111 or 112. The light intensity can be increased or the illumination conditions can be changed by adjusting the parameters of the illumination device. Alternatively or additionally, a cleaning can be activated by a cleaning module 130 in order to remove or at least reduce detected contamination on the field of view or on a illumination device 111. Alternatively, other activatable process executions 835 are possible, such as adjustments of the monitor parameters of a monitor 364. After the process execution (step 835), the process goes back to data acquisition 310 and starts the individual steps of data preparation (see step 311) and subsequent data evaluation (see step 124) again.

A plurality of databases can be stored in the memory 122 according to the device unit to be controlled. Preferably, there is at least one parameter database that is used for the process execution 135 when contaminated images are detected in the optimization query (step 825). Depending on the category of contaminated images, there are correspondingly adjusted cleaning parameters stored, such as the gas or liquid flushing quantity, the pressure, the pulse duration, the intervals between the pulses and the pulse-pause ratio or others.

The following criteria can be stored in the memory unit or in the self-learning module for parameter optimization. The main criterion for parameter assignment and optimization is cleanliness and image quality, i.e. the most complete possible removal of contamination. A secondary criterion for parameter assignment and optimization is the minimum cleaning time. The user should be minimally disturbed, analogous to a short windshield wiper movement. Another secondary criterion is the minimal fluid consumption, e.g. of liquids such as water or sodium chloride or other physiologically well-tolerated fluids. Finally, a minimal gas consumption can enter the parameter formation.

The self-learning module is designed in such a way that the parameter adjustment, such as a pressure increase or a pulse extension, is preferably carried out in such a way that the main criteria and secondary criteria are met with a high degree of probability. The type or procedure for optimization, ie the level and order of parameter changes, can be different for different image class contamination levels. This means that additional data sets or a further parameter database can be assigned in the memory 122 for the individual image classes or degrees of contamination. The method is based on probability considerations and the trained model is based on a stochastic model. Other possible models can serve as a basis for the parameter adjustment, such as an empirical model, which is based on measurement data from at least one sensor. The images themselves can also be trained by a suitable model to generate labels or markers for the raw data provided. In this way, labeled image data sets can facilitate and accelerate the training of the training model. In addition or as a correction, experts can still produce labeled images.

If the trained model is based on an open neural network, corrections can be made by experts. Through so-called labeling, an expert or other appropriately trained user can mark an existing image from the raw data with a label that indicates a need for optimization or with a symbol such as OK, which means that no optimization is necessary. These labeled images can then be used as training data for the model to be trained. In this case, at least one set of training data, preferably several sets of training data, can be prepared and then made available for learning by the model 322 to be trained to the machine learning device (see self-learning module 320, e.g. in FIG. 3). In particular, if the largest possible number of training images pre-labeled by experts are provided for each class, the learning phase of the model can be advantageously shortened,

The training data generated by machine learning is in turn stored in the reference database. The self-learning module 320 can form a trained model 322 through a neural network, such as a multi-layer neural network, which has image data with corresponding markings, characteristics or labels as an input layer and outputs classified images as an output layer. Such a neural network also has an intermediate layer and can, for example, use a convolutional neural network (CNN).

The preferably labeled training data are stored in the memory unit 122 and the trained model 322 of the self-learning module 320 learns using this training data 331. The model 322 trained with training data 331 carries out a determination of a quality value (see step 124) based on an image acquired by the image acquisition device 1115 (see step 310) or an image section of this image (see step 311) and then outputs the determination result (step 825). If the determination result or the quality value shows that no optimization is necessary or no contamination was detected, the process is restarted immediately or at specified intervals (see NO arrow to step 801).

If the decision step 825 resulted in a process execution 835 for optimizing the image, the optimization result can be evaluated with the self-learning module based on pixel evaluations with parameters such as color, resolution, light, contrast and/or sharpness. The pixels are preferably evaluated in such a way that organs or tissues to be examined can be distinguished from contamination. This can be done by comparing image data from the database in the memory 122 and is indicated by a double arrow between the respective device units 111, 130, 365 and 367 and the memory 122.

The disclosure can be applied to a large number of endoscopic systems established on the market in order to diagnose the respective examination object more precisely and reliably and to be able to continue working on the site as quickly as possible despite disruptions. The optimization method and optimization system according to the disclosure can make automated decisions in step 825 as to whether optimization is necessary and whether image optimization can be carried out by the controllable devices. For example, contaminant detection and cleaning can be automated. Fast cleaning results are particularly important in medical technology. The aim is to avoid any long-term disruption to the user and to eliminate any contamination or image disturbance as quickly as possible. The continuous provision of training data continuously improves the quality of the AI algorithms and the continuous parameter adjustments result in an optimal “tuning” of the process executions 835.

A software or computer program product for carrying out the image optimization process is configured to use image analysis algorithms to detect non-optimal images or disturbances such as image contamination with sufficient probability. Using image analysis, a quality value and existing image data (image history) or parameter data can be evaluated and compared with the latest image data and previous optimization results. In addition to a statistical model, the algorithms can use a stochastic model and issue control instructions based on corresponding marginal probabilities as to whether the current image can be optimized or not. In an open artificial network, existing data records can be corrected by experts in addition to automatic procedures, and the reference database in the memory 122 can be expanded to include new data records automatically or manually. Continuous semi-automatic or automatic checking of the optimization method increases safety for the user and thus for the person being examined using the endoscopic method.

The invention claimed is:

1. A method for image analysis and optimization of at least one image acquired at the distal end of an endoscope, wherein the method comprises the following steps:
acquiring image data by means of an image acquisition device of an endoscope;
receiving the image data by a control unit,
pre-analyzing at least a subset of the image data of one or more consecutive images by a control unit to determine at least one image structure;
determining a quality value by means of a training data-based, preferably self-learning, module by comparing the at least one determined image structure with image structures of a reference database stored in a memory unit, and
on the basis of the determined quality value, manually or automatically outputting control instructions from the control unit to a unit for activating image optimization.

2. The method according to claim 1, wherein the unit that can be activated for image optimization is a unit of the endoscope; and preferably comprises a cleaning module for cleaning at least one distal window by means of at least one fluid and the image optimization takes place via the activation of the cleaning module.

3. The method according to claim 1, wherein the quality value depends on the detected brightness of the image and/or contamination of the at least one distal window, wherein, in the step of determining the quality value by means of the self-learning module, a classification into contamination probabilities and/or degree of contamination takes place.

4. The method according to claim 1, wherein the self-learning module comprises a model with a neural network,
wherein the input data comprises the image data acquired by the image acquisition device, which data can be extracted as individual images, pixels and/or image sections; and
wherein the output data comprises the probability of contamination or blindness of the image acquisition device.

5. The method according to claim 1, wherein the training data comprise image structures stored in the memory unit and/or a collection of characteristics which are used to train a model of the self-learning module, wherein the trained model of the self-learning module is trained to classify the image data into the following contamination-dependent database classes:
non-contaminated images,
contaminated images which can be cleaned by a cleaning module;
and contaminated images or images which have characteristics which can be optimized by modules other than the cleaning module.

6. The method according to claim 5, wherein the self-learning module is based on machine learning, wherein a pre-classification is carried out by an expert or several persons and the subsequent quality value-dependent classification is carried out by a neural network;
optionally, algorithms derived from the training data for the model of the self-learning module can be checked and corrected by at least one expert.

7. The method according to claim 1, wherein the self-learning module has a model with a neural network based on machine learning or deep learning, wherein the neural network is selected from the group comprising:
an open neural network;
a closed neural network;
a single-layer neural network;
a multi-layer feedforward network with hidden layers;
a feedback neural network; and
combinations thereof.

8. The method according to claim 1, wherein the at least one image structure represents at least one object to be examined that is depicted in the acquired image; and
wherein contamination detection, deterioration or smoke detection is based on a change in the image structure.

9. The method according to claim 1, wherein, in the step of pre-analyzing, a division of one of the acquired images into image sections is carried out and/or a division into individual regions based on pixels is carried out on the basis of analysis values, wherein the quality value can be determined for each of the image sections and/or regions.

10. The method according to claim 2, wherein the cleaning module is designed to activate a regionally targeted cleaning depending on a definable weighting of regions and/or depending on a permissible percentage of contamination,
wherein preferably the region of the distal window that can be assigned to the image center can be cleaned in a targeted manner.

11. The method according to claim 10, wherein, after activation of the cleaning module, a pulse of a fluid jet with a duration of a few milliseconds (“ms”) to a maximum of 1000 ms and under high pressure of a maximum of 3 bar is directed specifically for cleaning at least a part of the distal window of the image acquisition device and/or at one or more distal illumination windows of light sources.

12. The method according to claim 1, wherein the unit that can be activated for image optimization is selected from the group comprising:
one or more light sources of the endoscope for changing the illuminance;
at least one filter for optimizing a contrast;
autofocus system for adjusting the sharpness of the image;
color spectrum change unit;
end lens heating;
monitor;
user-dependent and/or endoscope- or light guide-dependent customizable software;
room illumination of the operating room; and
combinations thereof.

13. The method according to claim 1, wherein the determination of a quality value comprises a blindness value due to smoke or condensation, wherein the control unit compares acquired characteristics for smoke or condensation with characteristics of a collection of characteristics stored in the reference database.

14. The method according to claim 2, wherein the method further comprises a control instruction to a smoke evacuator and/or an insufflator or a flushing pump in order to optimize the image by means of smoke evacuation; and/or
to optimize the fluid management in the body cavity to be examined with the endoscope by means of the insufflator or the flushing pump as a function of fluid flows during smoke evacuation or cleaning.

15. The method according to claim 2, wherein the intracorporeal pressure in the body cavity is measured with a pressure sensor, and the control unit controls the intracorporeal pressure in an event- and/or time-controlled manner at least during the duration of a cleaning by means of a control of the at least one pump or a control of a pressure regulator such that the intracorporeal pressure does not exceed a predetermined maximum limit value.

16. The method according to claim 5, wherein, the cleaning module is designed to adapt the type of cleaning by changing the cleaning parameters depending on the database classes and/or a degree of contamination,
wherein one or more cleaning parameters are selected from a group comprising:
type of fluid, fluid volume, fluid volumes, fluid velocity, pressure, pulse duration, number of pulses, pulse-pause ratio and/or total cleaning duration.

17. The method according to claim 1, wherein the self-learning module comprises a cleaning control algorithm based on an image analysis history, which algorithm checks the cleaning effectiveness of previous cycles in order to select cleaning parameters adapted to the history, preferably based on stochastic approximation algorithms, in order to achieve the highest probability to obtain an image with sufficiently good image quality.

18. The method according to claim 16, wherein the adjustment of the cleaning parameters is carried out by means of a deep learning model of the self-learning module or based on a step-by-step increase or by means of maximum settings.

19. The method according to claim 1, wherein the self-learning module for building up the reference database receives training data of image structures of characteristic images of objects to be examined, preferably organs or tissue structures, and/or of smoke via an input interface automatically or by an input via a user.

20. The method according to claim 1, wherein the following initialization steps take place before or during the acquisition of image data:
providing at least one further database with technical data of usable image acquisition devices and/or cleaning modules;
comparing the provided image acquisition device with the technical data and detecting the provided image acquisition device; and, depending on the detected image acquisition device, transmitting stored cleaning parameters to the cleaning module for cleaning activation.

21. The method according to claim 20, wherein the method further comprises the following steps:
starting a detection routine to detect structures of a body cavity or a technical cavity, preferably a region or object to be examined, based on the image data, and
continuing the method based on a positive result of the detection routine or
interrupting the method based on a negative result of the detection routine.

22. The method according to claim 2, further comprising a monitoring routine with the following steps:
after cleaning by means of the cleaning module, analyzing the image data by a control unit;
comparing the image data with stored parameters for a positive cleaning result; and
depending on the cleaning result, interrupting the cleaning or repeating the cleaning.

23. The method according to claim 2, further comprising:
adaptation of the cleaning to the degree of image deterioration;
wherein in the case of severe image deterioration, cleaning with liquid is activated and cleaning parameters defined depending on the endoscope provided are provided; and
wherein in the case of slight image deterioration or sufficient probability of image improvement after liquid cleaning, cleaning with gas is activated and cleaning parameters defined depending on the endoscope provided are provided for drying.

24. A control system for image optimization of at least one image acquired at the distal end of an endoscope, wherein the system comprises:
an image acquisition device;
a control unit with a self-learning module in communication with the image acquisition device for receiving the image data and a memory unit;
wherein the self-learning module is designed to determine at least one quality value based on training data by comparing at least one specific image structure with image structures of a reference database stored in the memory unit, and
wherein the control unit is designed to automatically output control instructions to a unit of the endoscope or an external unit for activating image optimization based on the determined quality value.

25. A computer program product comprising instructions which, when executed by a computer, cause the computer to perform the steps of the method according to claim 1.

26. A trained model trained with training data, wherein the training data comprise image structures stored in the memory unit and/or a collection of characteristics which are used to train a model of the self-learning module, wherein the trained model of the self-learning module is trained to classify the image data into the following contamination-dependent database classes:
non-contaminated images,
contaminated images which can be cleaned by a cleaning module;
and contaminated images or images which have characteristics which can be optimized by modules other than the cleaning module, and wherein the trained model is designed to carry out the steps of the method according to claim 1.
